(19) United States
(12) Patent Application Publication
(10) Pub. No.: US 2020/0189936 A1
(43) Pub. Date: Jun. 18, 2020

(54) FLUID STERILIZING DEVICE

(71) Applicant: Stanley Electric Co., Ltd., Tokyo (JP)

(72) Inventors: Hiroyuki Kato, Tokyo (JP); Hideaki Tanaka, Tokyo (JP)

(21) Appl. No.: 16/714,266

(22) Filed: Dec. 13, 2019

(30) Foreign Application Priority Data
Dec. 13, 2018 (JP) 2018-233082

(51) Int. Cl. C02F 1/32; A61L 2/10

(52) U.S. Cl. CPC C02F 1/325; A61L 2/10; C02F 2201/328; C02F 2301/022; C02F 2201/3222

(57) ABSTRACT

A fluid sterilizing device (1) includes barrel portion (5) having a channel where fluid to be sterilized flows; inlet (6a) formed on one end portion side of the barrel portion (5); outlet (7a) formed on the other end portion side of the barrel portion (5); a light source (3) that emits ultraviolet light toward the fluid; and a rectifier (12) mounted inside the channel and having a cylindrical through hole. The rectifier (12) includes inner circumferential region Rᵢₙ expanding from its center in the diameter direction of the channel, and outer circumferential region Rₒᵤₜ expanding outside the inner circumferential region Rᵢₙ. The ratio (t/d)ₒᵤₜ of the panel thickness t relative to the diameter d of each through hole in the outer circumferential region Rₒᵤₜ is larger than the ratio (t/d)ᵢₙ of the panel thickness t relative to the diameter d of each through hole in the inner circumferential region Rᵢₙ.

### BACKGROUND OF THE INVENTION

### 1. Field of the Invention

The present invention relates to a fluid sterilizing device that sterilizes fluid flowing in a channel with ultraviolet light.

### 2. Description of the Related Art

In recent years, the sterilizing effect of ultraviolet light (wavelengths of 240 to 380 nm) is utilized for germicidal lamps of food storages or medical equipment. In addition, a device that emits ultraviolet light from an ultraviolet LED to fluid flowing in a channel to sterilize the fluid so that the sterilized fluid is used as cleaning water is well known.

For example, Patent Document 1 mentioned below discloses a sterilizing device including a plurality of light emitting devices, a substrate, a rod lens, a window, an enclosure, and a rectifier. The enclosure is shaped like a box whose inside is divided into a processing chamber, a light source chamber, a cooling channel, a first discharge channel, and a second discharge channel.

The enclosure has a rectifier that is mounted at the inlet of the enclosure to rectify the flow of fluid flowing in through the inlet. With rectification, the fluid flowing into the processing chamber is made into a laminar flow, so that the ultraviolet light can be conveyed farther than in a case where the fluid flows in a turbulent state in the processing chamber. This also prolongs a period of time with the ultraviolet light acting on the fluid, whereby the accumulated irradiation amount of ultraviolet light upon the fluid can be increased.

### RELATED ART DOCUMENT

### Patent Document

Patent Document 1: Japanese Patent Laid-Open Publication No. 2017-051290

### SUMMARY OF THE INVENTION

### Technical Problem

Unfortunately, although the fluid is made into a laminar flow with a simple rectifier in the sterilizing device disclosed in Patent Document 1 as the diameter of the inlet is the same as that of the upstream wall of the processing chamber, a more complicated flow will be generated in an enclosure that has an inlet whose diameter is different from that of the processing chamber. This leads to a problem in that the effect produced by a rectifier is lessened.

The present invention has been conceived in view of the above, and aims to provide a fluid sterilizing device that uniformizes the flow rate distribution of fluid to reduce variation in the irradiation period of ultraviolet light upon the fluid to thereby enhance efficiency in sterilization.

### Solution to Problems

A fluid sterilizing device according to a first aspect of the present invention includes an enclosure having a channel where fluid to be sterilized flows in the axial direction; an inlet formed on the side of one end portion of the enclosure such that the fluid flows in the channel along the axial direction; an outlet formed on the side of the other end portion of the enclosure so that the fluid flows out through the outlet; a light source configured to emit ultraviolet light via ultraviolet light transmissive material toward the fluid; and a rectifier mounted inside the channel on the side of the one end portion of the enclosure so as to be perpendicular to the axis, the rectifier having a plurality of cylindrical through holes, wherein the rectifier includes an inner circumferential region expanding from the center of the rectifier in the diameter direction of the channel, and an outer circumferential region expanding outside the inner circumferential region, and the ratio (t/d)ₒᵤₜ of the panel thickness t of the rectifier relative to the diameter d of each through hole in the outer circumferential region is larger than the ratio (t/d)ₒᵤₜ of the panel thickness t of the rectifier relative to the diameter d of each through hole in the inner circumferential region.

According to the present invention, the fluid, which is a target of sterilization, flows through the inlet into the enclosure having a channel and flows out through the outlet. As the light source emits ultraviolet light toward the fluid flowing in the channel via ultraviolet light transmissive material, the fluid is sterilized

In the above, the fluid is rectified by the rectifier provided inside the channel on the side of one end portion of the enclosure. The ratio (t/d) of the panel thickness t relative to the diameter d of a through hole is different between the inner circumferential region and the outer circumferential region of the rectifier. In particular, as the ratio (t/d)ₒᵤₜ relevant to the outer circumferential region is larger than the ratio (t/d)ᵢₙ relevant to the inner circumferential region, a flow of the fluid toward the pipe wall of the channel, the fluid having flowed out through the through hole in the inner circumferential region, is generated. This uniformizes the flow rate distribution of the fluid between the pipe wall and the middle of the pipe (around the axis). Hence, this device can reduce variation in the irradiation period of ultraviolet light upon the fluid so that the efficiency in sterilization can be enhanced.

In the fluid sterilizing device according to the first aspect of the present invention, preferably, the panel thickness t of the rectifier is uniform, and the diameter dᵢₙ of each through hole formed in the inner circumferential region is larger than the diameter dₒᵤₜ of each through hole formed in the outer circumferential region.

With this structure, in the case where the panel thickness t of the rectifier is uniform, when the diameter dᵢₙ of a through hole in the inner circumferential region is larger than the diameter dₒᵤₜ of a through hole in the outer circumferential region, the ratio (t/d)ₒᵤₜ relevant to the outer circumferential region is resulted larger than the ratio (t/d)ᵢₙ relevant to the inner circumferential region. As such, setting a different diameter d of a through hole between the inner circumferential region and the outer circumferential region of the rectifier can readily uniformize the flow rate distribution of the fluid.

Also, in the fluid sterilizing device according to the first aspect, the diameters d of the through holes of the rectifier may be the same, and the panel thickness tₒᵤₜ in the outer circumferential region may be larger than the panel thickness tᵢₙ in the inner circumferential region.

With this structure, in the case where the diameters d of the through holes are the same, when the panel thickness tₒᵤₜ relevant to the outer circumferential region is larger than the panel thickness tᵢₙ relevant to the inner circumferential region, the ratio (t/d)ₒᵤₜ relevant to the outer circumferential region is resulted larger than the ratio (t/d) ᵢₙ relevant to the inner circumferential region. As such, setting a different panel thickness t of the rectifier between the inner circumferential region and the outer circumferential region of the rectifier as well can uniformize the flow rate distribution of the fluid.

In the fluid sterilizing device according to the first aspect of the present invention, preferably, the rectifier has a concave shape whose panel thickness t becomes smaller as it goes closer to the center of the rectifier.

In the case where the diameters d of the through holes are the same, as the panel thickness t needs to be different between the inner circumferential region and the outer circumferential region of the rectifier, the rectifier has a concave shape whose panel thickness t becomes smaller as it goes toward its center. This shape can make the ratio (t/d)ₒᵤₜ relevant to the outer circumferential region larger than the ratio (t/d)ᵢₙ relevant to the inner circumferential region.

In the fluid sterilizing device according to the first aspect of the present invention, preferably, the inlet has a cylindrical shape that is coaxial with the channel, and the inner circumferential region of the rectifier has a round shape whose diameter is equal to the diameter D of the inlet.

With this structure, the diameter of the inner circumferential region (round shape) of the rectifier is equal (including being substantially equal) to the diameter Dᵢₙ of the inlet (cylindrical shape), which is coaxial with the channel. With the above, this device can have a ratio (t/d) relevant to the inner circumferential region of the rectifier adapted to the components of the fluid flowing straight to the rectifier from the inlet, to thereby uniformize the flow rate distribution of the fluid.

In the fluid sterilizing device according to the first aspect of the present invention, preferably, the ratio (t/d) of the panel thickness t of the rectifier relative to the diameter d of each through hole is less than 0.65.

The ratio (t/d) has a correlation with a coefficient of an outflow angle, or a ratio of the outflow angle relative to the inflow angle at which the fluid flows into the rectifier. In particular, in the case where the ratio (t/d) is less than 0.65, a flow of the fluid toward the pipe wall of the channel, the fluid having passed through at the middle (in the inner circumferential region) of the rectifier, is generated. With this flow, this device can enhance an effect of uniformizing the flow rate distribution of the fluid.

In the fluid sterilizing device according to the first aspect of the present invention, preferably, the inlet and the channel each have a cylindrical shape, and the ratio (Dᵢₙ/D) of the diameter D of the inlet relative to the diameter D of the channel is equal to 0.46 or greater and less than 1.

The flow rate distribution of fluid depends also on the ratio (Dᵢₙ/D), or a ratio of the diameter Dᵢₙ of the inlet (cylindrical shape) relative to the diameter D of the channel (cylindrical shape). With a rectifier in use, when the ratio (Dᵢₙ/D) has a value equal to 0.46 or greater or less than 1, a flow rate distribution of a stable turbulence is resulted, and this device can uniformize the flow rate distribution of the fluid.

In the fluid sterilizing device according to the first aspect of the present invention, preferably, the light source emits ultraviolet light in a direction perpendicular to the direction in which the fluid flows.

For example, disposition of a light source in the outer circumferential region of the channel enables emission of ultraviolet light in a direction perpendicular to the direction in which the fluid flows. With the above, this device can efficiently sterilize the fluid flowing in a long channel shaped like a straight pipe.

In the fluid sterilizing device according to the first aspect of the present invention, the light source may emit ultraviolet light in a direction parallel to the direction in which the fluid flows.

For example, disposition of the light source on an end portion of the channel enables emission of ultraviolet light in a direction parallel to the direction in which the fluid flows. With the above, this device can efficiently sterilize the fluid having approached to the end portion of the channel.

In the fluid sterilizing device according to the first aspect of the present invention, preferably, the light source is a cool cathode tube whose axial direction extends in the axial direction of the channel.

With use of a cool cathode tube whose axial direction extends along the axial direction of the channel, this device can efficiently sterilize the fluid flowing in a channel shaped like either a straight pipe or an L-shape.

A fluid sterilizing device according to a second aspect of the present invention includes an enclosure having a channel where fluid to be sterilized flows in the axial direction; an inlet formed on the side of one end portion of the enclosure such that the fluid flows in the channel along the axial direction; an outlet formed on the side of the other end portion of the enclosure so that the fluid flows out through the outlet; a light source configured to emit ultraviolet light via ultraviolet light transmissive material toward the fluid; and a rectifier mounted inside the channel on the side of the one end portion of the enclosure so as to be perpendicular to the axis, the rectifier having a plurality of cylindrical through holes, wherein the ratio (t/d) of the panel thickness t of the rectifier relative to the diameter d of each through hole becomes larger as it goes farther away from the center of the rectifier.

According to the present invention, the fluid is rectified by the rectifier provided inside the channel on the side of one end portion of the enclosure, and the ratio (t/d) of the panel thickness t relative to a through hole d becomes larger as it goes farther away from the center of the rectifier. With the above, a flow of the fluid toward the pipe wall of the channel, the fluid having flowed out, in particular, through a through hole at the center (in the inner circumferential region) of the rectifier, is generated Thus, as the flow rate distribution of the fluid is uniformized between the pipe wall and the middle of the pipe (near the axis), this device can reduce variation in the irradiation period of ultraviolet light upon the fluid, so that the efficiency in sterilization can be enhanced.

A fluid sterilizing device according to a third aspect of the present invention includes an enclosure having a channel where fluid to be sterilized flows in the axial direction; an inlet formed on the side of one end portion of the enclosure such that the fluid flows in the channel along the axial direction; an outlet formed on the side of the other end portion of the enclosure so that the fluid flows out through the outlet; a light source configured to emit ultraviolet light via ultraviolet light transmissive material toward the fluid; and a rectifier mounted inside the channel on the side of the one end portion of the enclosure so as to be perpendicular to the axis, the rectifier having a plurality of cylindrical through holes, wherein a condition that the ratio (t/d) of the panel thickness t of the rectifier relative to the diameter d of each through hole is less than 0.65 is satisfied.

According to the present invention, the fluid is rectified by the rectifier provided inside the channel on the side of one end portion of the enclosure, and a condition that the ratio (t/d) of the panel thickness t relative to the diameter d of a through hole is less than 0.65 is satisfied. With the above, a flow of the fluid toward the pipe wall of the channel, the fluid having flowed out, in particular, through a through hole at the center of the rectifier, is generated. Thus, as the flow rate distribution of the fluid is uniformized between the pipe wall and the middle of the pipe (near the axis), this device can reduce variation in the irradiation period of ultraviolet light, so that the efficiency in sterilization can be enhanced.

In the fluid sterilizing device according to the third aspect of the present invention, preferably, the inlet has a cylindrical shape that is coaxial with the channel, the diameter of a round inner circumferential region of the rectifier is equal to the diameter D₁ of the inlet, and each through hole formed in the inner circumferential region satisfies the condition.

With this structure, the diameter of the inner circumferential region (round shape) of the rectifier is equal (including being substantially equal) to the diameter Dᵢₙ of the inlet (cylindrical shape), which is coaxial with the channel, and further, the ratio (t/d) relevant to the inner circumferential region of the rectifier is less than 0.65. With the above, a part of the components of the fluid flowing straight from the inlet to the rectifier flows toward the pipe wall of the channel after passing through the through hole. Therefore, this device can uniformize the flow rate distribution of the fluid.

### BRIEF DESCRIPTION OF THE DRAWINGS

FIG. 1 is a perspective view of an entire fluid sterilizing device according to the present invention (a first embodiment);

FIG. 2 is a cross sectional view of the fluid sterilizing device in FIG. 1 along the line II-II;

FIG. 3 is a front view and a side view of a rectifier (1);

FIG. 4 is a diagram relevant to the kind and thickness ratio of a rectifier;

FIG. 5 illustrates results of simulations for flow rate distributions;

FIG. 6 is a diagram relevant to a coefficient of an inflow angle;

FIG. 7 is a diagram relevant to the relationship between a thickness ratio and a coefficient of an inflow angle;

FIG. 8A is a front view and a side view of a rectifier (2);

FIG. 8B is a front view and a side view of a rectifier (3);

FIG. 8C is a front view and a side view of a rectifier (4);

FIG. 9 is a diagram relevant to the relationship between a thickness ratio and the irradiation amount of ultraviolet light;

FIG. 10 is a diagram relevant to the relationship between the ratio between an inlet diameter and a channel diameter and the ratio between an average flow rate and a maximum flow rate;

FIG. 11 is a perspective view of an entire fluid sterilizing device according to the present invention (a second embodiment);

FIG. 12 is a perspective view of an entire fluid sterilizing device according to the present invention (a modified example of the second embodiment);

FIG. 13 is a perspective view of an entire fluid sterilizing device according to the present invention (a third embodiment); and

FIG. 14 is a cross sectional view of the fluid sterilizing device in FIG. 13 along the line X-X.

### DESCRIPTION OF THE PREFERRED EMBODIMENTS

Embodiments of a fluid sterilizing device according to the present invention will now be described.

### First Embodiment

FIG. 1 is a perspective view of an entire fluid sterilizing device according to a first embodiment of the present invention. The fluid sterilizing device 1 is a device that irradiates fluid flowing in a channel with ultraviolet light to thereby sterilize the fluid. The fluid sterilizing device 1 is used for water purifiers or industrial circulation devices.

The fluid sterilizing device 1 has a substrate 4 on which a light source (not illustrated) is mounted and a channel, and includes, for example, a barrel portion 5 constituting a sterilizing unit that sterilizes fluid, a reducer 6 having an inlet 6a for fluid, a reducer 7 having an outlet 7a for the fluid, and a reflector 8 disposed surrounding the barrel portion 5.

When the reflector 8 is mounted on the substrate 4, the light source is disposed in the state of being fit in the opening of the reflector 8, as to be described late in detail. Here, as the barrel portion 5 is made of quartz, or an ultraviolet light transmissive material, the ultraviolet light emitted from the light source passes through the barrel portion 5 and sterilizes the fluid.

As illustrated, a metal heat sink 11 is disposed on the side of the rear surface of the substrate 4 (the side free from the light emitting surface of the light source). A connecter 9 connects a wire 9a and the light source.

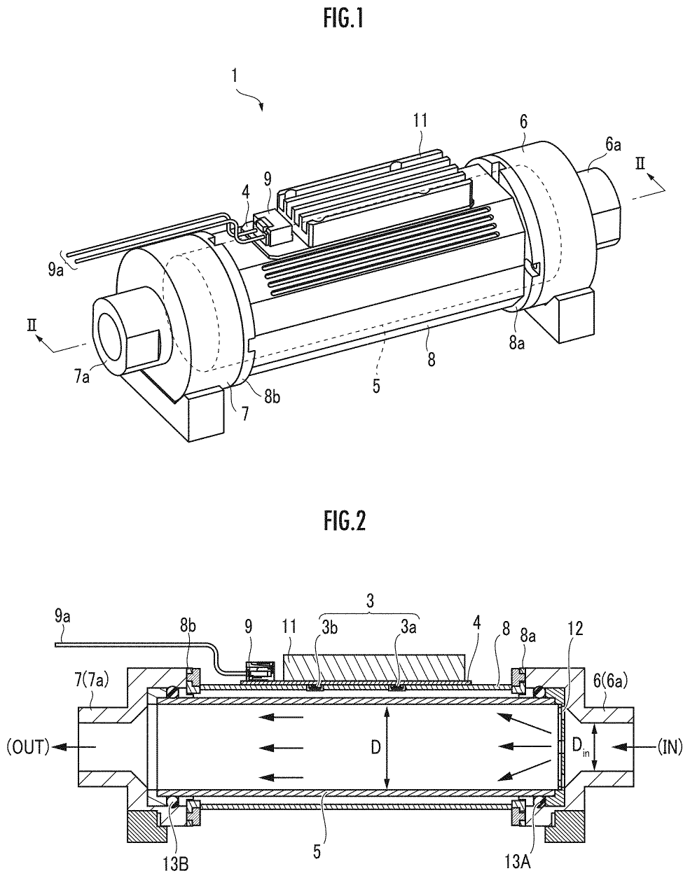

FIG. 2 is a cross sectional view of the fluid sterilizing device 1 in FIG. 1 along the line II-II.

A light source 3 includes an LED 3a and an LED 3b, and is mounted on the side of the front surface of the substrate 4 (the side with the light emitting surface of the light source). The ultraviolet light emitted from the light source 3 has wavelengths that have sterilizing effect or decompose chemical materials, which are wavelengths in the range of 240 to 380 nm, for example.

The substrate 4 is desirably made of metal, such as copper or aluminum, which is superior in heat discharge characteristics. The light source 3 is fed with power via the wire 9a, the connecter 9, and the substrate 4. The substrate 4 abuts on the reflector 8 on the side of the front surface of the substrate 4, and is securely screwed.

On the side of the rear surface of the substrate 4, the heat sink 11 for discharging heat is provided. This enables efficient discharge of heat generated from the light source 3.

The barrel portion 5 (corresponding to the “enclosure” according to the present invention) is shaped like a cylindrical straight pipe whose diameter is 48 mm (the inner diameter D of 44 mm) and the length of which channel (the sterilizing unit) is 200 mm. Fluid that is a target of sterilization flows in the longitudinal axial direction of the barrel portion 5. As the barrel portion 5 is a quartz pipe, the ultraviolet light emitted from the light source 3 passes through the barrel portion 5.

On an end portion of the reflector 8 in the axial direction (on the right side in the drawing), a flange 8a is formed, where the reducer 6 (the angle of divergence of 65°) is screwed (refer to FIG. 1). Fluid flows in through the cylindrical inlet 6a (the inner diameter Dᵢₙ of 27 mm). As the O-ring 13A is disposed to thereby seal between the reducer 6 and the barrel portion 5, invasion of the fluid into the reflector 8 is prevented.

On the other end portion of the reflector 8 in the axial direction (on the left side in the drawing), a flange 8b is formed, where the reducer 7 (the angle of divergence of) 65° is screwed. The fluid flows out through the cylindrical outlet 7a (the inner diameter of 27 mm). As illustrated, the respective center axes of the inlet 6a, the channel of the barrel portion 5, and the outlet 7a are common (coaxial), and the amount of fluid is, for example, about 10 (L/min).

Similarly, an O-ring 13B is disposed to thereby seal between the reducer 7 and the barrel portion 5, so that invasion of the fluid into the reflector 8 is prevented. Note that the O-rings 13A, 13B can deteriorate through exposure to ultraviolet light even though the O-rings 13A, 13B are made of fluorine-based material. As the O-rings 13A, 13B, however, are disposed at positions that are rarely irradiated with the ultraviolet light, the O-rings 13A, 13B can be saved from deterioration.

The fluid having flowed in through the inlet 6a passes through a rectifier 12 provided on an end portion of the reducer 6 opposite from the inlet 6a, and reaches the channel of the barrel portion 5. The rectifier 12 is a panel made of metal or fluorine resin and having two or more cylindrical through holes penetrating therethrough in the axial direction of the barrel portion 5. As the fluid passes through the rectifier 12, the flow rate of the fluid is averaged when the fluid flows into the channel of the barrel portion 5.

The fluid having reached the channel of the barrel portion 5 is exposed to the ultraviolet light emitted from the light source 3, which is fit in the opening of the reflector 8, and diffused by the reflector 8. With the above, the fluid is uniformly irradiated with the ultraviolet light. This improves performance in sterilization.

Referring to FIG. 3 to FIG. 5, results of simulation for determining flow rate distributions of the fluid while changing the kind of the rectifier will now be described. Note that the flow rate distribution refers to a distribution of flow rates on a plane perpendicular to the channel here.

In the fluid sterilizing device 1 (refer to FIG. 1), assuming that the amount of fluid is 10 (L/min), the average flow rate is 0.11 (m/s) (a turbulence is resulted as the Raynolds number Re is about 4,800, or being larger than 4,000). As a light source, eight deep ultraviolet LEDs (265 nm, 50 mW) in total are disposed at respective positions with an interval of 90° around the reflector 8 and away by ±20 mm from the substantial middle of the channel in the axial direction (z=110 mm with the position of the rectifier z=0).

FIG. 3 is a front view and a side view of a rectifier 12A, or one of the rectifiers used here. The rectifier 12A is of a 60° zigzag-type with a hole diameter (diameter) d of each through hole of 2.0 mm, a pitch p of 3.0 mm, an opening rate β of 0.403, and a panel thickness t of 1.0 mm, 1.3 mm, or 3.0 mm. The rectifier 12A is disposed on the right end portion (z=0) of the barrel portion 5 (refer to FIG. 2).

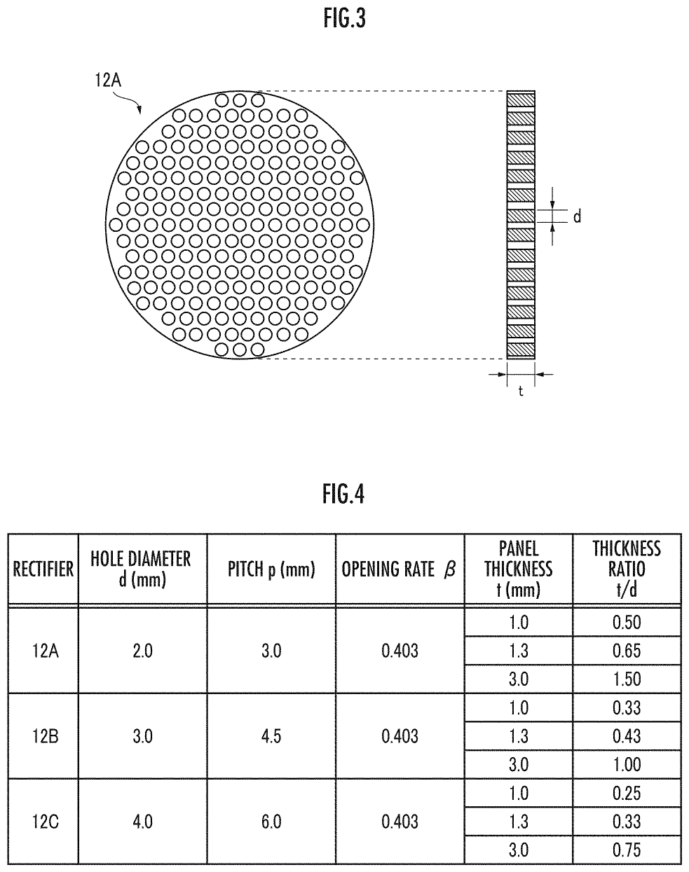

FIG. 4 shows three kinds of panel thicknesses t and thickness ratios (t/d) of the rectifier 12A, the thickness ratio (t/d) being a ratio of a panel thickness t relative to the hole diameter d. As a rectifier other than the rectifier 12A, a rectifier 12B and a rectifier 12C are prepared. The rectifier 12B has a hole diameter d of each through hole of 3.0 mm, a pitch p of each hole of 4.5 mm, and an opening rate β of 0.403. The rectifier 12C has a hole diameter d of a through hole of 4.0 mm, a pitch p of 6.0 mm, and an opening rate β of 0.403. Each of the rectifiers 12B, 12C has three kinds of panel thicknesses t.

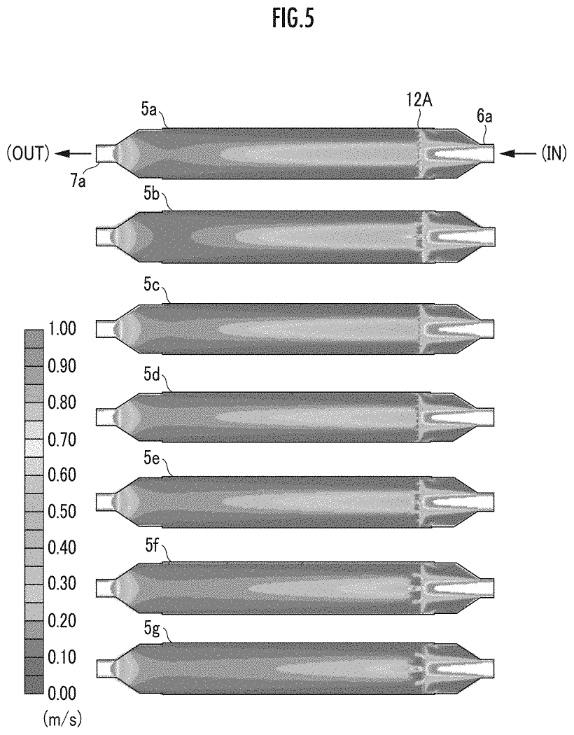

FIG. 5 illustrates the results of simulation for flow rate distributions. For this simulation, general-purpose fluid analysis software ANSYS FLUENT (Ver.16.2) is used, and k-e is employed as a turbulence model. A hexagonal mesh is used as a computational grid. As to illumination distribution, a general purpose optical simulation software ASAP is used.

The irradiation amount of ultraviolet light upon the fluid is calculated based on the ultraviolet sensitivity of MS2 (phage), and a simulation through particle tracking (the number of particles: about 8,000), based on an assumption that an ultraviolet light transmittance UVT of water is 95% (UVT=95%) and the reflection rate R of the reflector 8 is 90% (R=90%).

FIG. 5 illustrates flow rate distributions, from top to bottom, beginning with one with the larger thickness ratio, including the barrel portion 5a with the thickness ratio (t/d)=1.5 (a maximum thickness ratio), the barrel portion 5b with the thickness ratio (t/d)=0.75, the barrel portion 5c with the thickness ratio (t/d)=0.65, the barrel portion 5d with the thickness ratio (t/d)=0.5, the barrel portion 5e with the thickness ratio (t/d)=0.43, the barrel portion 5f with the thickness ratio (t/d)=0.33, and the barrel portion 5g with the thickness ratio (t/d)=0.25 (a minimum thickness ratio).

In the example of the barrel portion 5a, the flow rate is 0.05 to 0.15 (m/s) near the pipe wall, 0.50 to 0.60 (m/s) at the middle of the pipe (inside the pipe wall, near the axis), and 0.80 to 0.90 (m/s) at a position immediately before the fluid flows into the rectifier 12A and a position immediately after the fluid has flowed into the outlet 7a.

As illustrated in FIG. 5, a portion with a faster flow rate is prolonged farther (toward the outlet 7a) in the axial direction of the barrel portion 5 with respect to a larger thickness ratio (t/d). In the case of a faster flow rate, however, the irradiation amount of ultraviolet light upon the fluid can be insufficient, which is not a preferred state. Thus, it is known that the uniformity in flow rate distribution between the pipe wall and the middle of the pipe is higher when the thickness ratio (t/d) is smaller, which is a preferred state.

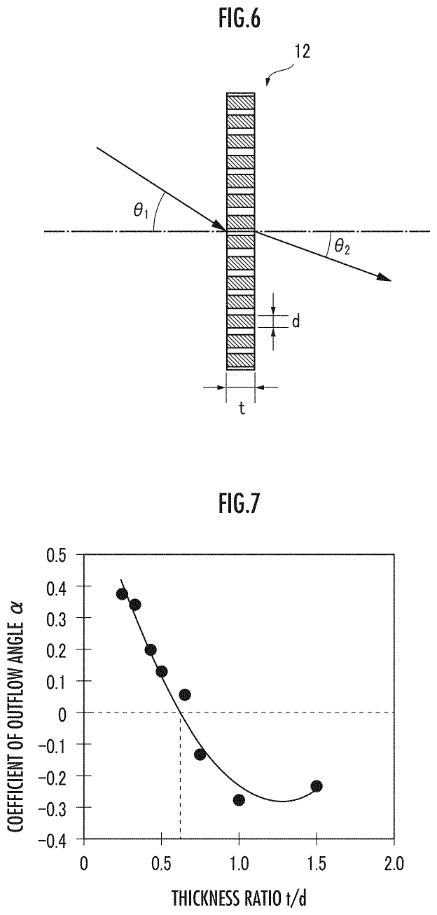

Referring to FIG. 6 and FIG. 7, a phenomenon in which the fluid flows into the rectifier will now be described.

As illustrated in FIG. 6, in general, the fluid having flowed into the rectifier 12 at an inflow angle θ₁ flows out from the rectifier 12 at an outflow angle θ₂. In the above, the inflow angle θ₁ and the outflow angle θ₂ hold a proportional relationship to each other, which is given by the expression (1) below given.

θ₂=αθ₁ (1)

wherein α is referred to as a coefficient of an outflow angle.

The coefficient of the outflow angle a varies depending on the value of the thickness ratio (t/d) of the rectifier 12. As illustrated in FIG. 7, the coefficient of the outflow angle a decreases as the thickness ratio increases, and becomes 0 with respect to the thickness ratio (t/d) of about 0.65. Also, it is known from the expression (1) that, when the coefficient of the outflow angle a becomes zero, the outflow angle θ₂ becomes zero (α→0 leads to θ₂→0), and that with the coefficient of the outflow angle α of zero, or α=0, the fluid flows out at the right angle relative to the rectifier 12, not depending on the value of the inflow angle θ₁.

Further, the coefficient of the outflow angle a takes a negative value as the thickness ratio increases. That is, in the area with the coefficient of the outflow angle a being a positive value, the fluid having passed through the rectifier 12 spreads toward the pipe wall of the barrel portion 5, and in the area with the coefficient of the outflow angle α being a negative value, the fluid converges toward the middle of the barrel portion 5.

Consequently, as illustrated in FIG. 5, the fluid having passed through a rectifier with a large thickness ratio (for example, the barrel portion 5a with t/d=1.5) converges toward the middle of the pipe of the barrel portion 5, so that a portion with a faster flow rate is prolonged farther. In contrast, as the fluid having passed through a rectifier with a small thickness ratio (for example, the barrel portion 5g with t/d=0.25) spreads toward the pipe wall, the flow rate diverges immediately after the fluid passes through the rectifier. It is known from the above-described result that a thickness ratio (t/d) of the rectifier of less than 0.65 is preferred.

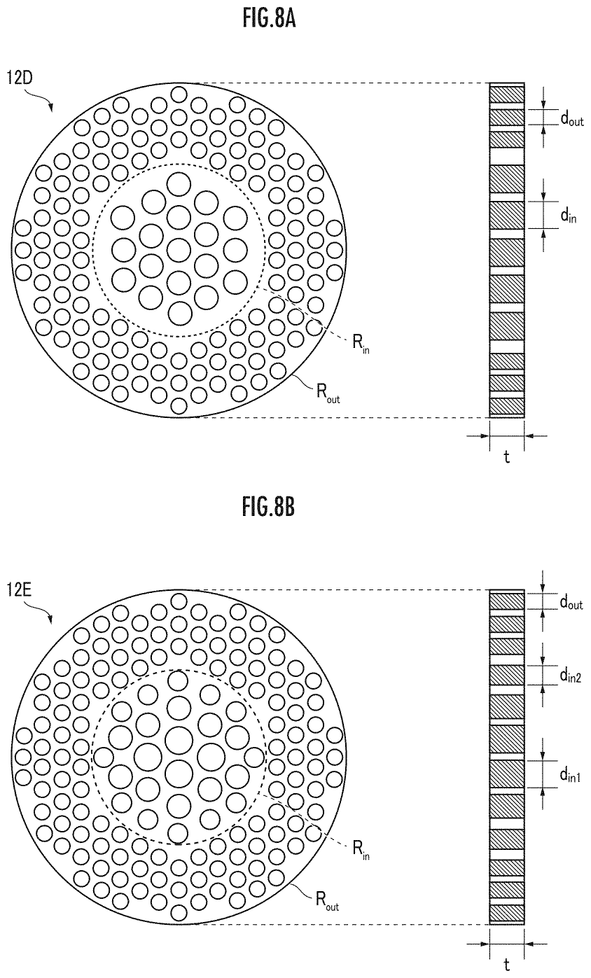

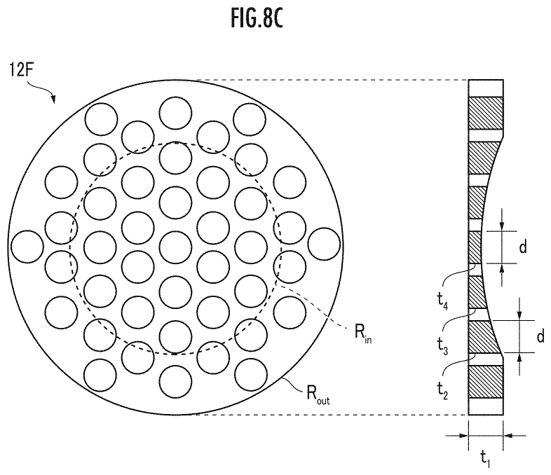

Referring to FIG. 8A to FIG. 8C, a structure of a rectifier that takes the above-described simulation result into account will now be described.

FIG. 8A illustrates a rectifier 12D having a uniform panel thickness t, an inner circumferential region Rᵢₙ expanding from the center of the rectifier 12D in the diameter direction of the channel, and an outer circumferential region Rₒᵤₜ expanding outside the inner circumferential region Rᵢₙ. The diameter of the inner circumferential region Rᵢₙ is substantially equal to the diameter Dᵢₙ of the inlet.

As illustrated, the hole diameter dᵢₙ of each through hole formed in the inner circumferential region Rᵢₙ and the hole diameter dₒᵤₜ of each through hole formed in the outer circumferential region Rₒᵤₜ hold a relationship of dᵢₙ>dₒᵤₜ. Thus, the thickness ratio holds a relationship of (t/dᵢₙ)<(t/dₒᵤₜ).

Although the thickness ratios (t/dᵢₙ) and (t/dₒᵤₜ) both have values less than 0.65, as the thickness ratio (t/dᵢₙ) has a smaller value, the fluid having flowed to around the center of the rectifier 12D tends to flow toward the pipe wall of the barrel portion, which uniformizes the flow rate distribution.

FIG. 8B illustrates a rectifier 12E having a uniform panel thickness t, in which the hole diameters d of the through holes become smaller as it goes farther away from the center of the rectifier 12E. As illustrated, through holes each having a hole diameter dᵢₙ₁ and through holes each having a diameter dᵢₙ₂ (dᵢₙ>dᵢₙ₂) are present in the inner circumferential region Rᵢₙ of the rectifier 12E, while through holes each having a hole diameter dₒᵤₜ (dᵢₙ₂>dₒᵤₜ) are present in the outer circumferential region Rₒᵤₜ.

With the above, the thickness ratio holds a relationship of (t/dᵢₙ₁)<(t/dᵢₙ₂)<(t/dₒᵤₜ). Although the thickness ratios (t/dᵢₙ₁), (t/dᵢₙ₂), and (t/dₒᵤₜ) all have values less than 0.65, as the thickness ratio becomes smaller as it goes closer to the center of the rectifier 12E, the fluid having flowed in around the center of the rectifier 12E tends to flow toward the pipe wall of the barrel portion, which as well uniformizes the flow rate distribution.

Note that the hole diameter d is not limited to three kinds, and four or more kinds are applicable. Also, through holes having two or more different hole diameters d may be formed not only in the inner circumferential region Rᵢₙ but also in the outer circumferential region Rₒᵤₜ.

As illustrated in FIG. 8A and FIG. 8B, in the case where the inner circumferential region Rᵢₙ and the outer circumferential region Rₒᵤₜ are defined in a rectifier having a uniform panel thickness t and through holes having different hole diameters d are formed in the respective regions, a condition that the thickness ratio (t/dᵢₙ) has a value less than 0.65 can be satisfied as to the hole diameter dᵢₙ of the through holes formed at least in the inner circumferential region Rₒᵤₜ.

With additional condition that the diameter of the inner circumferential region Rᵢₙ of the rectifier is equal to the diameter Dᵢₙ of the inlet, at least the fluid having flowed in around the center of the rectifier tends to flow toward the pipe wall of the barrel portion. With the above, even if the hole diameter dₒᵤₜ of the through hole in the outer circumferential region Rₒᵤₜ does not satisfy the condition that the thickness ratio (t/dₒᵤₜ) has a value less than 0.65, the rectifier can produce an effect of uniformizing the flow rate distribution to some extent.

FIG. 8C illustrates a rectifier 12F having through holes having the same hole diameter d, an inner circumferential region Rᵢₙ expanding from the center of the rectifier 12F in the diameter direction of the channel, and an outer circumferential region Rₒᵤₜ expanding outside the inner circumferential region Rᵢₙ.

The panel thickness t₁ of the rectifier 12F is maximum in the outer circumferential region Rₒᵤₜ, and the panel thickness becomes smaller as it goes closer to the center of the rectifier 12F in the inner circumferential region Rᵢₙ of the rectifier 12F. In other words, the panel thickness becomes smaller in the order of the panel thicknesses t₂, t₃, t₄.

With the above, the thickness ratio holds a relationship of (t₁/d)<(t₂/d)<(t₃/d)<(t₄/d). Although the thickness ratios of (t₁/d), (t₂/d), (t₃/d), and (t₄/d) all have values less than 0.65, as the thickness ratio has smaller values as it goes closer to the center of the rectifier 12F, the fluid having flowed in around the center of the rectifier 12F tends to flow toward the pipe wall of the barrel portion, which as well uniformizes the flow rate distribution.

Here again, the diameter of the inner circumferential region Rᵢₙ of the rectifier 12F is substantially equal to the diameter Dᵢₙ of the inlet. Note that, in the case where a rectifier has a convex shape, like the rectifier 12F, the panel thickness t results in different between the peripheral side and the central side of one through hole. Thus, a stepped structure that is stepped down toward the center of the rectifier 12F is applicable.

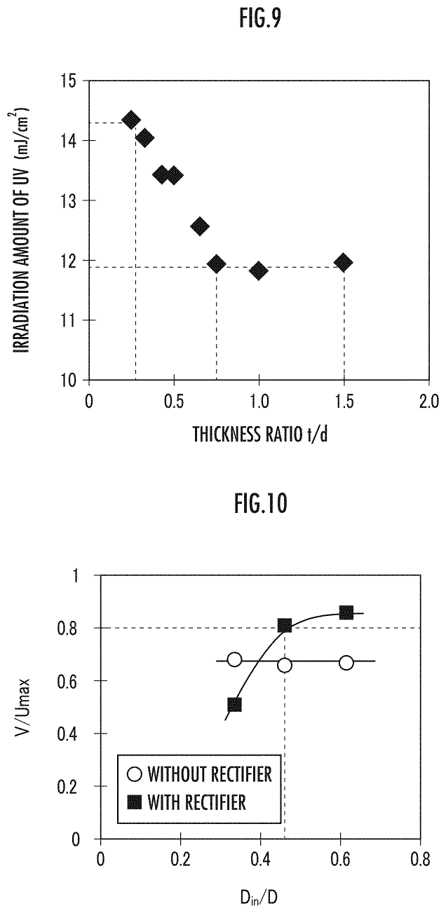

Referring to FIG. 9 and FIG. 10, a structure of a channel that takes the above-described simulation results into account is now described.

FIG. 9 illustrates the relationship between the above-described thickness ratio and the irradiation amount of ultraviolet light upon the fluid. Change in flow rate distribution due to change in thickness ratio appears as change in the irradiation amount of ultraviolet light upon the fluid. In particular, when the thickness ratio (t/d) is changed from 0.25 to 0.75, the irradiation amount of ultraviolet light upon the fluid decreases from 14.3 (mJ/cm²) to 11.9 (mJ/cm²).

When the thickness ratio (t/d) is increased to set the thickness ratio (t/d) to 1.5, it is resulted that the irradiation amount of ultraviolet light becomes constant at 11.9 (mJ/cm²). This proves that the thickness ratio (t/d) of less than 0.65 is preferred also in view of the irradiation amount of ultraviolet light upon the fluid.

A ratio between the average flow rate (V) and a maximum flow rate (Uₘₐₓ) is determined, using the rectifier 12C illustrated in FIG. 4 (the thickness ratio t/d=0.25), while changing the diameter Dᵢₙ of the inlet 6a. As the diameter Dᵢₙ becomes larger, such as from 14.7 mm, 20.2 mm, to 27.0 mm, the value of V/Uₘₐₓ becomes larger, and the value of V/Uₘₐₓ becomes 0.8 or greater with the diameter D of 20.2 mm or greater. That is, it is known that the flow rate distribution is uniformized with a single rectifier 12C. Note that, in contrast to a flow rate distribution of a laminar flow, a flow rate distribution disordered by turbulence is averaged into a distribution similar to that of a uniform flow. It is confirmed in an experiment that V/Uₘₐₓ takes a value of 0.5 in the state of a laminar flow, and of 0.8 or greater in the state of turbulence. Thus, with the V/Uₘₐₓ of 0.8 or greater, the flow rate distribution can be described as substantially uniform.

FIG. 10 illustrates dependency of the value of V/Uₘₐₓ (an average value) of the barrel portion 5 (an area where irradiation with ultraviolet light is possible Z=10 to 210 mm) on the ratio between the inlet diameter (Dᵢₙ) and the channel diameter (D). As illustrated, it is resulted that the value of V/Uₘₐₓ does not change in the absence of a rectifier. In contrast, in the presence of a rectifier (t/d=0.25), a flow rate distribution with the V/Uₘₐₓ having a value of 0.8 or greater is obtained with Dᵢₙ/D of 0.46 or greater.

With the above, it is known that Dᵢₙ/D having a value of 0.46 or greater and less than 1 is preferred, and that, in a fluid sterilizing device under this condition, provision of a single rectifier with the thickness ratio (t/d) of less than 0.65 on the side of the inlet enables formation of a uniform flow rate distribution.

### Second Embodiment

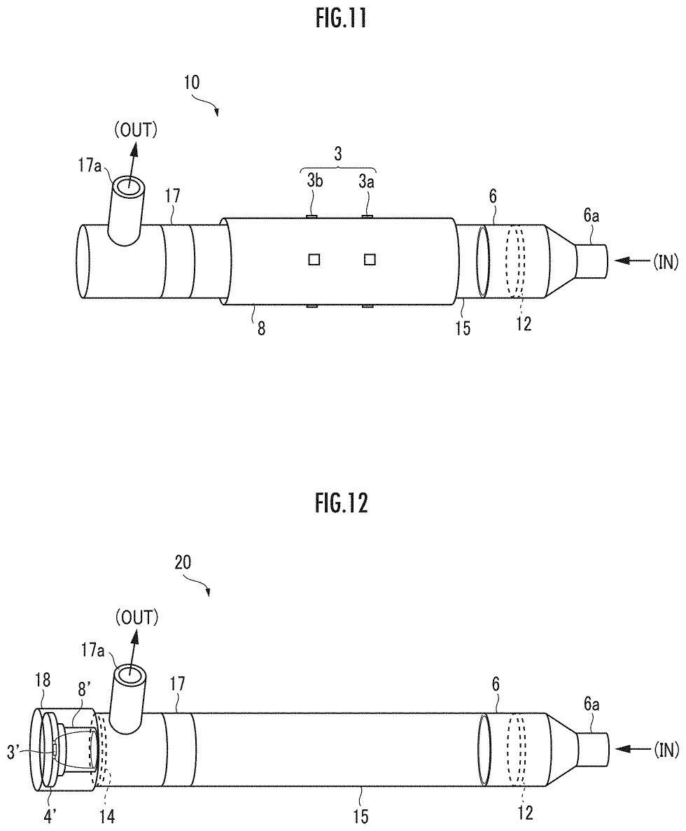

Referring to FIG. 11 and FIG. 12, a second embodiment of the fluid sterilizing device according to the present invention will now be described. Note that the same structure as that in the above-described embodiment will be hereinafter given the same reference numeral and its description is omitted.

FIG. 11 illustrates a fluid sterilizing device 10 having a channel, and including, for example, a barrel portion 15 that makes a sterilizing unit for sterilizing fluid, the reducer 6 having the inlet 6a for fluid, an outflow device 17 having an outlet 17a for the fluid, and the reflector 8 disposed so as to surround the circumference of the barrel portion 5. Being simply illustrated here, the light source 3 is mounted on the reflector 8 in the state of being mounted on a substrate (refer to FIG. 1).

The barrel portion 15 is shaped like a cylindrical straight pipe whose diameter is 48 mm (the inner diameter D is 44 mm), and the length of which channel is 200 mm. Fluid to be sterilized flows in the longitudinal axial direction of the barrel portion 15. As the barrel portion 15 is made of quartz, or an ultraviolet light transmissive material, the ultraviolet light emitted from the light source 3 passes through the barrel portion 15 so that the fluid is irradiated with the ultraviolet light to be thereby sterilized.

The reducer 6 is disposed on one end portion (on the right side in the drawing) of the barrel portion 15 in the axial direction. The fluid flows in through the cylindrical inlet 6a (the inner diameter D is 20.2 mm). Note that the angle of divergence of the inlet 6a is 54°.

The outflow device 17 is mounted on the other end portion (on the left side in the drawing) of the barrel portion 15 in the axial direction. The fluid flows out through the cylindrical outlet 7a (the inner diameter of 20.2 mm). The amount of fluid is, for example, about 10 (L/min). As such, the channel may have an L-shaped structure.

The fluid having flowed in through the inlet 6a passes through the rectifier 12 disposed on an end portion of the reducer 6 opposite from the inlet 6a to reach the channel of the barrel portion 15. As the fluid passes through the rectifier 12, the flow rate distribution is uniformized between the pipe wall and the middle of the pipe (near the axis) of the barrel portion 15.

The fluid having reached the channel of the barrel portion 15 is exposed to the ultraviolet light emitted from the light source 3 (LED 3a, LED 3b), which is fit in the opening of the reflector 8, and diffused by the reflector 8. With the above, the fluid is uniformly irradiated with the ultraviolet light, which improves the performance in sterilization.

Alternatively, like the fluid sterilizing device 20 illustrated in FIG. 12, the positions of the light source and the reflector may be changed such that a light source module device 18 is disposed to the left of the outflow device 17 (on the other end portion of the barrel portion 15, opposite from the inlet 6a). In the above, the light source module device 18 includes a light source 3′, a substrate 4′ for the light source 3′, and a reflector 8′ all being held therein. In addition, a quartz window 14 is provided between the outflow device 17 and the light source module device 18.

One light source 3′ is mounted on the side of the front surface of the substrate 4′. The substrate 4′ is desirably made of metal, such as copper or aluminum, which is superior in heat discharge characteristics. The light source 3′ is fed with power via the substrate 4′. On the side of the rear surface (on the opposite side from the light emitting surface of the light source 3′) of the substrate 4′, a heat sink for discharging heat may be disposed.

On the side of the front surface of the substrate 4′, the reflector 8′ is disposed so as to surround the light source 3′. The reflector 8′ is a spheroidal or paraboloidal reflection mirror. The ultraviolet light emitted from the light source 3′ is reflected on the inner surface of the reflector 8′ to pass through the quartz window 14 to proceed toward the channel of the barrel portion 15. With the above, the fluid having reached near the outlet 17a of the outflow device 17 is irradiated with the ultraviolet light.

As the light source 3′ of the fluid sterilizing device 20 emits ultraviolet light in a direction parallel to the direction in which the fluid flows (an end face emission type), the barrel portion 15 may not be made of an ultraviolet light transmissive material. For example, the barrel portion 15 may be made of stainless, and the inner wall of the barrel portion 15 may be coated with ultraviolet light reflecting material. This allows the ultraviolet light emitted from the light source 3′ to reach a position far from the light source 3′, which improves efficiency in sterilization.

Although the channels of the fluid sterilizing device 10 and the fluid sterilizing device 20 are L-shaped, a U-shaped structure in which an inlet and an outlet are both disposed perpendicular to the channel (in the circumferential direction of the barrel portion) is applicable.

### Third Embodiment

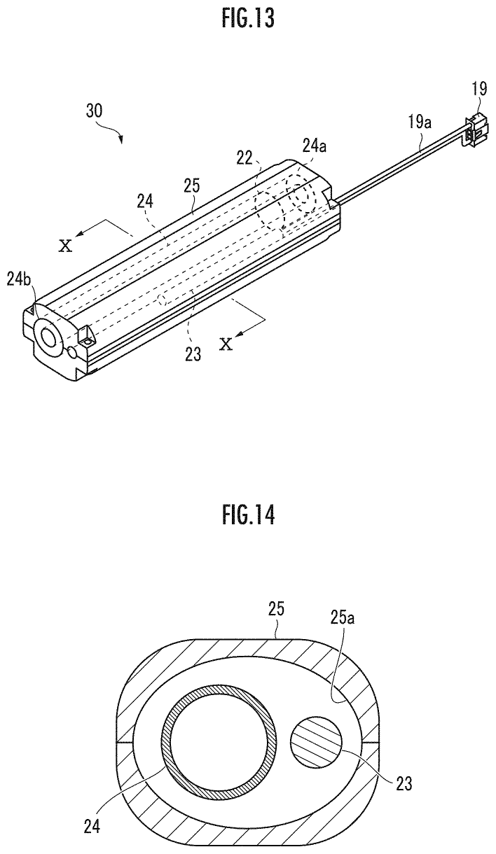

Finally, referring to FIG. 13 and FIG. 14, a fluid sterilizing device of an outside emission type according to a third embodiment of the present invention will be described.

As illustrated in FIG. 13, the fluid sterilizing device 30 includes a UV cool cathode tube 23 that makes a light source and a water flow pipe 24 where fluid flows, both being held in a barrel portion 25. The UV cool cathode tube 23 is a pillar-shaped or U-shaped lamp that emits ultraviolet light, and is fed with power via a connecter 19 and a wire 19a. The UV cool cathode tube 23 is disposed such that its axial direction extends along the water flow pipe 24.

Fluid flows in the channel through an inlet 24a of the water flow pipe 24, and flows out through an outlet 24b. As a rectifier 22 is disposed on the way (near the inlet 24a) of the channel, the flow rate distribution is uniformized by the rectifier 22

FIG. 14 is a cross sectional view of the fluid sterilizing device 30 in FIG. 13 along the line X-X.

As illustrated, the UV cool cathode tube 23 and the water flow pipe 24 are disposed adjacent to each other in the barrel portion 25. The fluid flows inside the water flow pipe 24. As the water flow pipe 24 is made of an ultraviolet light transmissive material, the ultraviolet light emitted from the UV cool cathode tube 23 passes through the water flow pipe 24, so that the fluid is irradiated with the ultraviolet light and thereby sterilized.

Although the inside of the barrel portion 25 and the outside of the water flow pipe 24 are hollow, as the inside wall of the barrel portion 25 is coated with ultraviolet light reflecting material, the inside wall functions as a reflector. With the above, the fluid is irradiated from every direction of the water flow pipe 24 with the ultraviolet light emitted from the UV cool cathode tube 23.

Although the fluid sterilizing device 30 is of a single light type having one UV cool cathode tube 23 held therein, the fluid sterilizing device 30 may be of a double light type having UV cool cathode tubes that are disposed so as to hold the water flow pipe 24 therebetween or of a multiple-light type having cool cathode tubes that are disposed so as to hold the water flow pipe 24 in three or more directions. Although the barrel portion 25 is shaped like a straight pipe, an L-shape is applicable.

Note that the above-described embodiments are mere examples, and can be arbitrarily changed depending on use. As the amount of flowing fluid differs depending on use, the dimension and shape of the barrel portion of the fluid sterilizing device are changeable.

Although an example in which the barrel portion has a cylindrical shape has been described in the above embodiments, this is not an exclusive shape. For example, the barrel portion may have a pillar shape whose cross section is round, oval, or polygonal.

In an arrangement in which a light source is disposed on one side of the channel, like the fluid sterilizing device 20, the flowing direction of the fluid is generally opposite from the emission direction of the ultraviolet light. Alternatively, the flowing direction may be matched with the emission direction. The numbers and directions of inlets and outlets and the number of ultraviolet LEDs, for example, are arbitrarily changeable.

In the case of a fluid sterilizing device whose inside wall of the barrel portion is made of polyvinyl chloride, the inside wall may be coated with an ultraviolet light reflecting material or an ultraviolet light absorbing material to prevent deterioration of the polyvinyl chloride due to ultraviolet light. As an ultraviolet light reflecting material, for example, aluminum or fluorine-based resin, such as polytetrafluoroethylene (PTFE), can be used. As an ultraviolet light absorbing material, stainless steel, for example, can be used.

### REFERENCE SIGNS LIST

1, 10, 20, 30 fluid sterilizing device, 3, 3′, 3a, 3b light source, 3a, 3B LED, 4, 4′ substrate, 5, 5a to 5g, 15, 25 barrel portion, 6, 7 reducer, 6a inlet, 7a outlet, 8, 8′ reflector, 8a, 8b flange, 9, 19 connecter, 9a, 19a wire, 11 heat sink, 12, 12A to 12F, 22 rectifier, 13A, 13B O-ring, 14 quartz window, 17 outflow device, 17a outlet, 18 light source module device, 23 UV cool cathode tube, 24 water flow pipe, 24a inlet, 24b outlet.

What is claimed is:

1. A fluid sterilizing device comprising:
an enclosure having a channel where fluid to be sterilized flows in a direction of an axis ;
an inlet formed on a side of one end portion of the enclosure such that the fluid flows in the channel along the direction of the axis;
an outlet formed on a side of another end portion of the enclosure so that the fluid flows out through the outlet;
a light source configured to emit ultraviolet light via ultraviolet light transmissive material toward the fluid; and
a rectifier mounted inside the channel on the side of the one end portion of the enclosure so as to be perpendicular to the axis, the rectifier having a plurality of cylindrical through holes,
wherein
the rectifier includes an inner circumferential region expanding from a center of the rectifier in a diameter direction of the channel, and an outer circumferential region expanding outside the inner circumferential region, and
a ratio (t/d)ₒᵤₜ of a panel thickness t of the rectifier relative to a diameter d of each through hole in the outer circumferential region is larger than a ratio (t/d)ᵢₙ of the panel thickness t of the rectifier relative to a diameter d of each through hole in the inner circumferential region.

2. The fluid sterilizing device according to claim 1, wherein
the panel thickness t of the rectifier is uniform, and
a diameter dᵢₙ of each through hole formed in the inner circumferential region is larger than a diameter dₒᵤₜ of each through hole formed in the outer circumferential region.

3. The fluid sterilizing device according to claim 1, wherein
the diameters d of the through holes of the rectifier are uniform, and
a panel thickness tₒᵤₜ in the outer circumferential region is larger than the panel thickness tᵢₙ in the inner circumferential region.

4. The fluid sterilizing device according to claim 3, wherein the rectifier has a concave shape whose panel thickness t becomes smaller as it goes closer to a center of the rectifier.

5. The fluid sterilizing device according to claim 1, wherein
the inlet has a cylindrical shape that is coaxial with the channel, and
the inner circumferential region of the rectifier has a round shape whose diameter is equal to a diameter Dᵢₙ of the inlet.

6. The fluid sterilizing device according to claim 1, wherein the ratio (t/d) of the panel thickness t of the rectifier relative to a diameter d of each through hole is less than 0.65.

7. The fluid sterilizing device according to claim 1, wherein the inlet and the channel each have a cylindrical shape, and
a ratio (Dᵢₙ/D) of a diameter Dᵢₙ of the inlet relative to a diameter D of the channel is equal to 0.46 or greater and less than 1.

8. The fluid sterilizing device according to claim 1, wherein the light source emits ultraviolet light in a direction perpendicular to a direction in which the fluid flows.

9. The fluid sterilizing device according to claim 1, wherein the light source emits ultraviolet light in a direction parallel to a direction in which the fluid flows.

10. The fluid sterilizing device according to claim 8, wherein the light source is a cool cathode tube whose axial direction extends in the direction of the axis of the channel.

11. A fluid sterilizing device comprising:
an enclosure having a channel where fluid to be sterilized flows in a direction of an axis;
an inlet formed on a side of one end portion of the enclosure such that the fluid flows in the channel along the direction of the axis;
an outlet formed on a side of another end portion of the enclosure so that the fluid flows out through the outlet;
a light source configured to emit ultraviolet light via ultraviolet light transmissive material toward the fluid; and
a rectifier mounted inside the channel on the side of the one end portion of the enclosure so as to be perpendicular to the axis, the rectifier having a plurality of cylindrical through holes,
wherein
a ratio (t/d) of a panel thickness t of the rectifier relative to a diameter d of each through hole becomes larger as it goes farther away from a center of the rectifier.

12. A fluid sterilizing device comprising:
an enclosure having a channel where fluid to be sterilized flows in a direction of an axis;
an inlet formed on a side of one end portion of the enclosure such that the fluid flows in the channel along the direction of the axis;
an outlet formed on a side of another end portion of the enclosure so that the fluid flows out through the outlet;
a light source configured to emit ultraviolet light via ultraviolet light transmissive material toward the fluid; and
a rectifier mounted inside the channel on the side of the one end portion of the enclosure so as to be perpendicular to the axis, the rectifier having a plurality of cylindrical through holes,
wherein
a condition that a ratio (t/d) of a panel thickness t of the rectifier relative to a diameter d of each through hole is less than 0.65 is satisfied.

13. The fluid sterilizing device according to claim 12, wherein
the inlet has a cylindrical shape that is coaxial with the channel,
a diameter of a round inner circumferential region of the rectifier is equal to a diameter Dᵢₙ of the inlet, and
each through hole formed in the inner circumferential region satisfies the condition.
